(19) United States
(12) Patent Application Publication
(10) Pub. No.: US 2008/0051348 A1
(43) Pub. Date: Feb. 28, 2008

(54) TREATMENT OF INFECTIONS AND OTHER DISORDERS

(75) Inventors: Allan L. Goldstein, Washington, DC (US); Jack Finkelstein JR., Chevy Chase, MD (US); David Crockford, Newburyport, MA (US)

Correspondence Address: ROTHWELL, FIGG, ERNST & MANBECK, P.C., 1425 K STREET, N.W., SUITE 800, WASHINGTON, DC, 20005, US

(73) Assignee: RegeneRx Biopharmaceuticals, Inc., Bethesda, MD (US)

(21) Appl. No.: 11/773,168

(22) Filed: Jul. 3, 2007

Related U.S. Application Data
(63) Continuation-in-part of application No. PCT/US06/00592, filed on Jan. 10, 2006.
(63) Continuation-in-part of application No. 10/503,555, filed on Oct. 21, 2004.
(60) Provisional application No. 60/642,520, filed on Jan. 11, 2005.
(60) Provisional application No. 60/354,250, filed on Feb. 6, 2002.
(60) Provisional application No. 60/421,038, filed on Oct. 25, 2002.

(51) Int. Cl. A61K 38/00; A61P 43/00

(52) U.S. Cl. 514/16; 514/17

(57) ABSTRACT

A method of treatment for treating, inhibiting, reducing or at least partly preventing respiratory or pulmonary microbial infection or gastrointestinal disorder of tissue of a subject, includes administering to a subject an effective amount of a composition including a polypeptide including thymosin β4 (TB4), an isoform of TB4, an N-terminal variant of TB4, a C-terminal variant of TB4, LKKTET or a conservative variant thereof, LKKTNT or a conservative variant thereof, TB4 sulfoxide, TB4^{ala}, Tβ9, Tβ10, Tβ11, Tβ12, Tβ13, Tβ14, Tβ15, gelsolin, vitamin D binding protein (DBP), profilin, cofilin, adsevertin, propomyosin, fincilin, depactin, Dnasel, vilin, fragmin, severin, capping protein, β-actinin, acumentin or a combination thereof, so as to inhibit the infection or disorder.

### CROSS-REFERENCE TO RELATED APPLICATION

This application is a continuation-in-part of International Application No. PCT/US2006/000592, filed Jan. 10, 2006, which claims the benefit of U.S. Provisional Application Ser. No. 60/642,520, filed Jan. 11, 2005.

This application also is a continuation-in-part of U.S. patent application Ser. No. 10/503,555, filed Oct. 21, 2004, which is a National Phase of International Application Serial No. PCT/US03/03455, filed Feb. 6, 2003, which claims the benefit of U.S. Provisional Application Ser. No. 60/354,250, filed Feb. 26, 2002 and U.S. Provisional Application Ser. No. 60/421,038, filed Oct. 25, 2002.

### BACKGROUND OF THE INVENTION

1. Field of the Invention

The present invention relates to the field of the treatment of microbial infections and gastrointestinal disorders.

2. Description of the Background Art

Treatment of microbial infections, bacterial, viral and fungal, can be difficult by conventional methods. These infections may include gastrointestinal infections (E. coli., H. pylon, VRE, etc.) abdominal infections (peritonitis, pancreatitis, gall bladder infections, etc.), surgical infections and osteomyelitis (bone infection).

Another example of microbial infection is anthrax. Anthrax is an infectious agent caused by Bacillus anthracis, a gram positive organism. It is primarily a disease of herbivores. Anthrax can affect many different vertebrates including humans. The symptoms of anthrax vary widely depending on the route of infection. Three forms of the disease commonly occur including a cutaneous form, a gastrointestinal form, and a pulmonary (inhalation) from. The pulmonary form of the disease is typically caused by inhalation of anthrax spores. The symptoms systemic anthrax can be mimicked in a number of animal models by the administration of virulence factors (endotoxins) which are responsible for the major pathologies, morbidity and mortality seen with anthrax. Once large amounts of anthrax toxins are produced within the body by bacteria, administration of antibiotics are usually ineffective. Anthrax induced pathologies mimic septic shock and the sudden death seen with other gram positive and gram negative bacterial infections such as multi-organ failure, edema and ARDS. In both animals and humans the anthrax induced pathologies also include marked elevation of TNFα, IL-1β, PAF and a number of other inflammatory cytokines. Also seen in anthrax induced septic shock is over production of reactive oxygen intermediates and an increase in aracidonic acid metabolites such as PGE, and thromboxane β₂ and disruption of the actin cytoskeleton.

A number of approaches have been reported to delay, prevent and/or treat exposure to anthrax. In the prevention area, a human vaccine is available by the effectiveness of the vaccine is unclear. The best treatment currently available is treatment with specific antibiotics such as ciprofloxaxin or doxycyclin. Antibiotics are effective if given at the very early stages of infection and are basically ineffective once the bacteria have had a chance to multiply rapidly producing lethal amounts of the deadly anthrax toxins. Of the three forms of anthrax, the most deadly form is pulmonary (Inhalation) anthrax which has a fatality rate of greater than 75% (even with appropriate antibiotic treatment). Anthrax produces a multi-component toxin that is assembled at the surface of host cells after infection. The lethal action of the anthrax toxins occurs in the cytoplasm of the host cells. The anthrax toxin is only one of many multi-subunit toxins that cause sever illness in humans. A major concern when treating bacterial infections with antibiotics is the appearance of increasing numbers of antibiotic resistant strains. In addition, once the anthrax bacillus has produced large amounts of exotoxins the antibiotics are basically ineffective.

Millions of Americans suffer from other gastrointestinal (GI) disorders such as colitis, ileitis, Crohn's disease, ulcerative colitis, colic, gingivitis, regional enteritis, ulcers, pouchitis, sclerosing, cholangitis, fistulae. The cause of many of these diseases is not known. However, they may have genetic roots or result from exposure to certain chemicals, pathogens, immune dysfunction, or foods during one's lifetime, or result from the normal aging of the human body. GI disorders occur in both men and women and can be acute or chromic, debilitating and life-threatening, and may occur anywhere within the GI tract, including but not limited to the mouth, throat, esophagus, stomach, small and large intestines, colon, and anus. People suffering from GI disorders may have a greatly diminished quality of life and suffer premature death.

A large number of therapeutic approaches to treatment have been reported for gastrointestinal disorders and disease, depending upon the location and the nature of the GI pathology. The treatments vary from surgical intervention, to dietary manipulations, to the use of a variety of drugs and biological agents. These agents include antibiotics, anti-virals, anti-inflammatory drugs, glucocorticoids, immunosuppressive drugs, monoclonal antibodies, antacids, anti-secretory drugs, anti-spasmodics, as well as a large number of others.

Numerous pharmaceutical, nutriceutical or cosmeceutical formulations have been proposed to treat the damage caused by microbial infections and gastrointestinal disorders.

Respiratory microbial infections, particularly respiratory bacterial infections, can be dangerous and even life-threatening.

Respiratory infections caused by Pseudomonas aeruginosa and other gram-negative bacteria occur almost exclusively in individuals with a compromised lower respiratory tract or a compromised systemic defense mechanism. Primary pneumonia occurs in patients with chronic lung disease and congestive heart failure. Bacteremic pneumonia commonly occurs in neutropenic cancer patients undergoing chemotherapy. Lower respiratory tract colonization of cystic fibrosis patients by mucoid strains of Pseudomonas aeruginosa and other gram-negative bacteria is common and difficult, if not impossible, to treat.

There remains a need in the art for methods of treatment for treating, inhibiting, reducing or at least partly preventing microbial infections and gastrointestinal disorders.

### SUMMARY OF THE INVENTION

In accordance with one aspect of the present invention, treatment of or inhibiting infections and gastrointestinal (GI) disorders, comprises administering to a subject in need of such treatment an effective amount of a composition comprising polypeptide comprising or consisting essentially of at least one of thymosin β4 (TB4), an isoform of TB4, an N-terminal variant of TB4, a C-terminal variant of TB4, LKKTET or a conservative variant thereof, LKKTNT or a conservative variant thereof, TB4 sulfoxide, Tβ4^{ala}, Tβ9, Tβ10, Tβ11, Tβ12, Tβ13, Tβ14, Tβ15, gelsolin, vitamin D binding protein (DBP), profilin, cofilin, adsevertin, propomyosin, fincilin, depactin, Dnasel, vilin, fragmin, severin, capping protein, β-actinin or acumentin.

### DETAILED DESCRIPTION OF THE INVENTION

In accordance with one aspect, a method of treatment for treating, inhibiting, reducing or at least partly preventing respiratory or pulmonary microbial infection of respiratory tissue of a subject, comprises administering to a subject in need of such treatment an effective amount of a composition comprising polypeptide comprising or consisting essentially of at least one of thymosin β4 (TB4), an isoform of TB4, an N-terminal variant of TB4, a C-terminal variant of TB4, LKKTET or a conservative variant thereof, LKKTNT or a conservative variant thereof, TB4 sulfoxide, Tβ4^{ala}, Tβ9, Tβ10, Tβ11, Tβ12, Tβ13, Tβ14, Tβ15, gelsolin, vitamin D binding protein (DBP), profilin, cofilin, adsevertin, propomyosin, fincilin, depactin, Dnasel, vilin, fragmin, severin, capping protein, β-actinin or acumentin, in said tissue, so as to inhibit said microbial infection.

Without being found to any specific theory, actin-sequestering peptides such as thymosin β4 (Tβ4) and other actin-sequestering peptides or peptide fragments containing amino acid sequence LKKTET, LKKTNT, or conservative variants thereof, promote treatment of microbial infections and gastrointestinal disorders. Without being bound to any particular theory, these peptides may have the capacity to promote repair, healing and prevention by having the ability to induce terminal deoxynucleotidyl transferase (a non-template directed DNA polymerase), to decrease the levels of one or more inflammatory cytokines or chemokines, and to act as a chemotactic and/or angiogenic factor for endothelial cells and thus treat damage caused by microbial infections and gastrointestinal disorders. Actin-sequestering peptides such as thymosin beta 4 (T [symbol] 4 or TB4) and other agents including actin-sequestering peptides or peptide fragments containing amino acid sequence LKKTET or LKKTNT or conservative variants thereof, promote reversal or at least partial prevention of respiratory infection of respiratory tissue.

In certain embodiments, a respiratory or pulmonary infection treated in accordance with the present invention is a bacterial infection, more preferably a gram-negative bacterial infection, such as a Pseudomonas aeruginosa respiratory infection.

A subject being treated in accordance with the present invention preferably is mammalian, most preferably human.

Thymosin 4 was initially identified as a protein that is up-regulated during endothelial cell migration and differentiation in vitro. Thymosin 4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration, vascularization and wound healing.

In accordance with one embodiment, the invention is a method of treatment of respiratory microbial infection of respiratory tissue of a subject, comprising administering to a subject in need of such treatment an effective amount of a composition comprising an agent, which may be a polypeptide comprising or consisting essentially of at least one of thymosin β4 (TB4), an isoform of TB4, an N-terminal variant of TB4, a C-terminal variant of TB4, LKKTET or a conservative variant thereof, LKKTNT or a conservative variant thereof, TB4 sulfoxide, Tβ4^{ala}, Tβ9, Tβ10, Tβ11, Tβ12, Tβ13, Tβ14, Tβ15, gelsolin, vitamin D binding protein (DBP), profilin, cofilin, adsevertin, propomyosin, fincilin, depactin, Dnasel, vilin, fragmin, severin, capping protein, β-actinin or acumentin. In accordance with other embodiments, the agent is other than thymosin beta 4 or Tβ4 sulfoxide.

International Application Serial No. PCT/US99/17282, incorporated herein by reference, discloses isoforms of T [symbol] 4 which may be useful in accordance with the present invention as well as amino acid sequence LKKTET and conservative variants thereof, which may be utilized with the present invention. International Application Serial No. PCT/GB99/00833 (WO 99/49883), incorporated herein by reference, discloses oxidized Thymosin [symbol] 4 which may be utilized in accordance with the present invention. Although the present invention is described primarily hereinafter with respect to [symbol] β4 and [symbol] β4 isoforms, it is to be understood that the following description is intended to be equally applicable to amino acid sequence LKKTET or LKKTNT, peptides and fragments comprising or consisting essentially of LKKTET or LKKTNT, conservative variants thereof having antimicrobial activity, and/or Tβ4 isoforms, analogues or derivatives, including N-terminal variants of Tβ4, C-terminal variants of Tβ4 and antagonists of Tβ4. The invention also may utilize oxidized Tβ4. The agent may be directly or indirectly antimicrobial.

In one embodiment, the invention provides a method of treatment for treating, inhibiting, reducing or at least partly preventing respiratory or pulmonary microbial infection of respiratory tissue of a subject, by contacting the tissue with an antimicrobial effective amount of a composition which contains an agent as described herein. As non-limiting examples, the tissue may be selected from respiratory tract or airway tissue of said subject. In one embodiment, the invention provides a method for healing damage caused by microbial infection in a subject by contacting an area to be treated with an effective amount of a composition as described herein. The contacting may be directly or systemically. Examples of direct administration include, for example, contacting the tissue, by direct application or inhalation, with a solution, lotion, salve, gel, cream, paste, spray, suspension, dispersion, hydrogel, ointment, or oil comprising an agent as described herein. Systemic administration includes, for example, intravenous, intraperitoneal, intramuscular injections or infusions of a composition containing an agent as described herein, in a pharmaceutically acceptable carrier such as water for injection. Systemic administration also includes, for example, intramuscular or subcutaneous injections, or inhalation, transdermal or oral administration of a composition. Enteral administration may include oral or rectal administration. A subject may be a mammal, preferably human.

In accordance with one embodiment, the invention is a method of treatment of damage associated with microbial infections comprising administering to a subject in need of such treatment an effective amount of a composition as described herein.

In one embodiment, the invention provides a method for treating bacterial infection comprising administering to a subject in need of such treatment, an effective amount of a composition as described herein.

In another embodiment, the invention provides a method for treating gastrointestinal infection. Common gastrointestinal infections include, but are not limited to Helicobacter pylori (H. pylon), Escherichia coli (E. coli.), vancomycin-resistant Enterococcus faecalis (VRE), and methicillin-resistant Staphylococcus aureus (MRSA). The composition may be delivered directly, or systemically by injection, orally, nasally, through suppository or enema, transdermally or any other suitable means.

In another embodiment, the invention provides a method for treating anthrax infection. Damage caused by anthrax infection includes septic shock, sudden death, multi-organ failure, edema, ARDS and inflammatory, degenerative, immunological damage. The composition can be applied alone or in combination with an antibiotic such as ciprofloxocin, doxycyclin, or penicillin. A therapeutically effective amount of the composition is applied to the site or systemically on a periodic basis during a course of therapy to reduce the mortality and morbidity effects of exposure to biological agents such as anthrax or to prevent such effects. The composition may also be delivered systemically by injection, orally, nasally, by inhalation or any other means to reduce the toxicity of pulmonary or gastrointestinal anthrax.

Another aspect of the invention is treatment of other Gastrointestinal disorders. In accordance with one embodiment, the invention is a method of treatment of damage associated with gastrointestinal disorders comprising administering to a subject in need of such treatment an effective amount of a composition comprising a gastrointestinal disorder-inhibiting polypeptide as described herein having gastrointestinal disorder inhibiting activity. This invention is applicable to inflammatory, ulcerative, degenerative, immunological and other injuries to and disorders of the gastrointestinal tract (from the mouth to the anus). These disorders occur due to genetic abnormalities, food intolerance, chemical exposure, aging, and microbial infections.

Gastrointestinal disorders to which the invention is applicable include, but are not limited to, gastrointestinal infections including bacterial, viral and fungal infections, disorders associated with environmental or iatrogenic abrasions, inflammations and other inflammatory disorders, immunological disorders, allergies including food allergies, Crohn's disease, ulcerative colitis, recurrent aphthous stomatitis (recurrent canker sores), ileitis, colic, gingivitis, regional enteritis, ulcers, pouchitis, sclerosing, cholangitis, fistulae and genetic abnormalities. They may result from exposure to certain chemicals, pathogens, immune dysfunction, or foods during one's lifetime, or result from the normal aging of the human body.

In one embodiment, the invention provides a method for healing damage caused by gastrointestinal disorders in a subject by contacting the affected tissue with a gastrointestinal disorder effective amount of a composition as described herein. The contacting may be topically, enterally or systemically. Examples of direct administration include, for example, contacting the affected tissue with a lotion, salve, gel, cream, paste, spray, suspension, dispersion, hydrogel, ointment, or oil comprising the polypeptide, alone or in combination with at least one agent that enhances the polypeptide penetration, or delays or slows release of the polypeptide into the area to be treated. Systemic administration includes, for example, intravenous, intraperitoneal, intramuscular or subcutaneous injections, or inhalation (orally or nasally), transdermal, suppository, enema or oral administration of a composition containing the polypeptide. A subject may be a mammal, preferably human.

The invention also is directed to a substance for use in manufacture of a medicament for treatment of microbial infections including anthrax, and gastrointestinal disorders, comprising a polypeptide as described herein.

Agents for use in the invention, as described herein, may be administered in any effective amount. For example, an agent as described herein may be administered in dosages within the range of about 0.0001-1,000,000 micrograms, more preferably in amounts within the range of about 0.1-5,000 micrograms, most preferably within the range of about 1-30 micrograms.

A composition in accordance with the present invention can be administered daily, every other day, every other week, every other month, etc., with a single application or multiple applications per day of administration, such as applications 2, 3, 4 or more times per day of administration.

Many T [symbol] 4 isoforms have been identified and have about 70%, or about 75%, or about 80% or more homology to the known amino acid sequence of T [symbol] 4. Such isoforms include, for example, Tβ4^{ala}, T [symbol] 9, [symbol] β10, T [symbol] 11, [symbol] β12, [symbol] β13, [symbol] β14 and T [symbol] 15. Similar to [symbol] β4, the T [symbol] 10 and T [symbol] 15 isoforms have been shown to sequester actin. T [symbol] 4, [symbol] β10 and T [symbol] 15, as well as these other isoforms share an amino acid sequence, LKKTET or LKKTNT, that appears to be involved in mediating actin sequestration or binding. For example, [symbol] β4 can modulate actin polymerization (e.g. [symbol] -thymosins appear to depolymerize F-actin by sequestering free G-actin). T [symbol] 4's ability to modulate actin polymerization may therefore be due to all, or in part, its ability to bind to or sequester actin via the LKKTET sequence. Thus, as with T [symbol] 4, other proteins which bind or sequester actin, or modulate actin polymerization, including T [symbol] 4 isoforms having the amino acid sequence LKKTET, are likely to be effective, alone or in a combination with [symbol] β4, as set forth herein.

Thus, it is specifically contemplated that known T [symbol] 4 isoforms, such as [symbol] β4^{ala}, T [symbol] 9, [symbol] β10, [symbol] β11, [symbol] β12, [symbol] β13, [symbol] β14 and [symbol] β15, as well as T [symbol] 4 isoforms not yet identified, will be useful in the methods of the invention. As such [symbol] β4 isoforms are useful in the methods of the invention, including the methods practiced in a subject. The invention therefore further provides pharmaceutical compositions comprising T [symbol] 4, as well as T [symbol] 4 isoforms T [symbol] 4^{ala}, T [symbol] 9, T [symbol] 10, T [symbol] 11, T [symbol] 12, [symbol] β13, T [symbol] 14 and T [symbol] 15, and a pharmaceutically acceptable carrier.

In addition, other agents or proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET or LKKTNT, for example, can similarly be employed in the methods of the invention. Such proteins may include gelsolin, vitamin D binding protein (DBP), profilin, cofilin, depactin, Dnasel, vilin, fragmin, severin, capping protein, [symbol] -actinin and acumentin, for example. As such methods include those practiced in a subject, the invention further provides pharmaceutical compositions comprising gelsolin, vitamin D binding protein (DBP), profilin, cofilin, depactin, Dnasel, vilin, fragmin, severin, capping protein, β-actinin and acumentin as set forth herein. Thus, the invention includes the use of an antimicrobial polypeptide comprising the amino acid sequence LKKTET or LKKTNT and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

[symbol] β4 has been localized to a number of tissue and cell types and thus, agents which stimulate the production of an LKKTET or LKKTNT peptide such as T [symbol] 4 or another agent as described herein, can be added to or comprise a composition to effect production an agent from a tissue and/or a cell. Such stimulating agents may include members of the family of growth factors, such as insulin-like growth factor (IGF-1), platelet derived growth factor (PDGF), epidermal growth factor (EGF), transforming growth factor beta (TGF- [symbol] ), basic fibroblast growth factor (bFGF), thymosin [symbol] 1 (T [symbol] 1) and vascular endothelial growth factor (VEGF). More preferably, the stimulating agent is transforming growth factor beta (TGF- [symbol] ) or other members of the TGF- [symbol] superfamily.

In accordance with one embodiment, subjects are treated with a stimulating agent that stimulates production in the subject of an agent as defined herein.

Additionally, other agents that assist in reduction of respiratory microbial infection of respiratory tissue may be added to a composition along with an agent as described herein. For example, and not by way of limitation, an agent as described herein alone or in combination can be added in combination with any one or more of the following agents: antibiotics, VEGF, KGF, FGF, PDGF, TGF [symbol] , IGF-1, IGF-2, IL-1, prothymosin [symbol] and/or thymosin [symbol] 1 in an effective amount.

The invention also includes a pharmaceutical composition comprising a therapeutically effective amount of an agent as described herein in a pharmaceutically acceptable carrier such as water for injection.

The actual dosage or reagent, formulation or composition that provides treatment may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable formulations may include an agent as described herein at a concentration within the range of about 0.001-50% by weight, more preferably within the range of about 0.01-0.1% by weight, most preferably about 0.05% by weight.

The therapeutic approaches described herein involve various routes of administration or delivery of an agent as described herein, including any conventional administration techniques (for example, but not limited to, direct administration, local injection, inhalation, or systemic administration), to a subject. The methods and compositions using or containing an agent as described herein may be formulated into pharmaceutical compositions by admixture with pharmaceutically acceptable non-toxic excipients or carriers.

The invention may include use of antibodies which interact with an agent as described herein. Antibodies which consist essentially of pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In yet another embodiment, the invention provides a method of treating a subject by administering an effective amount of stimulating agent which modulates gene expression. The term “modulate” refers to inhibition or suppression of expression when an agent as described herein is over expressed, and induction of expression when an agent as described herein is underexpressed. The term “effective amount” means that amount of stimulating agent which is effective in modulating gene expression of an agent as described herein. A stimulating agent which modulates gene expression of a response-inhibiting agent as described herein may be a polynucleotide, for example. The polynucleotide may be an antisense, a triplex agent, or a ribozyme. For example, an antisense directed to the structural gene region or to the promoter region of an agent as described herein may be utilized. The stimulating agent which modulates gene expression of an agent as described herein may also be a small interfering RNAs (siRNAs).

In another embodiment, the invention provides a method for utilizing compounds that modulate activity of an agent as described herein. Compounds that affect activity of an agent as described herein (e.g., antagonists and agonists) include peptides, peptidomimetics, polypeptides, chemical compounds, minerals such as zincs, and biological agents.

A method for screening for a stimulating agent as defined herein, comprises contacting a respiratory tissue exhibiting respiratory microbial infection, with a candidate compound; and measuring activity in said tissue of an LKKTET or LKKTNT peptide, wherein an increase of activity of said peptide in said tissue, compared to a level of activity of said peptide in a corresponding tissue lacking said candidate compound, indicates that said compound is capable of inducing said stimulating agent.

A further method of screening for a stimulating agent as defined herein, comprises contacting a respiratory tissue with a candidate compound, optionally microbially infecting the tissue, and measuring LKKTET or LKKTNT peptide activity in said tissue, wherein an increase of activity in said tissue, compared to a level of said LKKTET or LKKTNT peptide activity in a corresponding tissue lacking said candidate compound, indicates that said candidate compound is capable of stimulating production in said tissue of said peptide.

### EXAMPLE 1

The purpose of this study was to study the antimicrobial activity of peptides against common pathogens in vitro. Antimicrobial assays were performed to assess peptide activity against Pseudomonas aeruginosa. All tested samples (n=3) constitutively expressed mRNA for thymosin β-4 (Tβ4). This expression was not affected by IL-1β or TNF-α. None of the tested samples expressed hBD-4, -5, -6, HE2β1, histatins (Hist-1, -3), or liver-expressed antimicrobial peptides (LEAP-1, -2). Tβ4 (EC₅₀=26.5+1.6 μg/ml) was effective against Pseudomonas aeruginosa. The lowest dose where activity and/or protection was demonstrated against Pseudomonas aeruginosa was about 0.5-1 μg/ml.

### EXAMPLE 2

Antimicrobial assays were performed to assess peptide activity against Pseudomonas aeruginosa (PA), Staphylococcus aureus (PA), and Staphylococcus epidermidis (SE).

Tβ4 (EC₅₀=18.1±1.7 μg/ml) was effective against PA and was weakly effective against staphylococcal strains.

### EXAMPLE 3

Tβ4 showed antimicrobial activity against Staphylococcus aureus and Escherichia coli at concentrations of 5-20 n mol/ml, and increasingly dose-dependent activity against those microorganisms at concentrations of 50-200 n mol/ml.

1. A method of treatment for treating, inhibiting, reducing or at least partly preventing respiratory or pulmonary microbial infection of respiratory or pulmonary tissue of a subject, comprising administering to a subject in need of such treatment an effective amount of a composition comprising polypeptide comprising or consisting essentially of at least one of thymosin β4 (TB4), an isoform of TB4, an N-terminal variant of TB4, a C-terminal variant of TB4, LKKTET or a conservative variant thereof, LKKTNT or a conservative variant thereof, TB4 sulfoxide, β4^{ala}, Tβ9, Tβ10, Tβ11, Tβ12, Tβ13, Tβ14 and Tβ15, gelsolin, vitamin D binding protein (DBP), profilin, cofilin, adsevertin, propomyosin, fincilin, depactin, Dnasel, vilin, fragmin, severin, capping protein, β-actinin or acumentin, so as to inhibit said microbial infection.

2. The method of claim 1 wherein said infection is bacterial infection.

3. The method of claim 1 wherein said infection is by Pseudomonas aeruginosa.

4. The method of claim 1 wherein said polypeptide is thymosin beta 4 (Tβ4).

5. The method of claim 1 wherein said polypeptide is other than Tβ4.

6. The method of claim 5 wherein said polypeptide comprises amino acid sequence LKKTET, LKKTNT, KLKKTET, or LKKTETQ, an N-terminal variant of Tβ4, a C-terminal variant of Tβ4, an isoform of Tβ4, or oxidized Tβ4.

7. The method of claim 1 wherein said polypeptide is directly or indirectly antimicrobial.

8. The method of claim 7 wherein said polypeptide is indirectly antimicrobial, and said agent stimulates production of an LKKTET or LKKTNT peptide in tissue of said subject.

9. The method of claim 1 wherein said polypeptide is administered to said subject at a dosage within a range of about 1-30 micrograms.

10. The method of claim 1 wherein said polypeptide is administered by direct administration to said tissue, or by intravenous, intraperitoneal, intramuscular, subcutaneous, inhalation, transdermal or oral administration, to said subject.

11. The method of claim 1 wherein said composition is administered systemically.

12. The method of claim 1 wherein said composition is administered directly to said tissue.

13. The method of claim 12 wherein said composition is in the form of a solution, gel, creme, paste, lotion, spray, suspension, dispersion, salve, hydrogel or ointment formulation.

14. The method of claim 1 wherein said polypeptide is a recombinant or synthetic peptide.

15. The method of claim 1, wherein said infection is by gram-negative bacteria.
